(19) United States
(12) Patent Application Publication
(10) Pub. No.: US 2020/0102533 A1
(43) Pub. Date: Apr. 2, 2020

(54) CELL SORTING METHOD AND SYSTEM

(71) Applicant: Hung-Wei Wu, Tainan City (TW)

(72) Inventors: Hung-Wei WU, TAINAN CITY (TW); Cheng-Yuan HUNG, KAOHSIUNG CITY (TW); Hung-Yueh LAI, TAINAN CITY (TW)

(21) Appl. No.: 16/591,605

(22) Filed: Oct. 2, 2019

(30) Foreign Application Priority Data
Oct. 2, 2018 (TW) 107134843

(51) Int. Cl. C12N 5/00; C12N 5/071; C12N 5/073; B01L 3/00

(52) U.S. Cl. CPC C12N 5/0081; C12N 5/0682; C12N 5/0605; B01L 2300/18; B01L 2200/0652; B01L 2400/0424; B01L 3/50273

(57) ABSTRACT

A cell sorting method includes: obtaining a cervical sample of a pregnant mammal, the cervical sample including placental trophoblast cells and cervical cells; removing the mucus of the cervical sample; dispersing the placental trophoblast cells and the cervical cells; centrifuging the cervical sample to remove the supernatant of the cervical sample; and using a dielectrophoretic chip to perform sorting on the cervical sample, so as to sort out the placental trophoblast cells from the cervical cells.

### RELATED APPLICATIONS

This application claims priority to Taiwan Application Serial Number 107134843, filed on Oct. 2, 2018, which is herein incorporated by reference.

### BACKGROUND

### Technical Field

The invention relates to a cell sorting method and a system, and more particularly to a cell sorting method and a system capable of sorting out placental trophoblast cells from a cervical sample.

### Description of Related Art

Prenatal fetal chromosome test is used to determine whether a fetus has a congenital disease. Common prenatal fetal chromosome testing is mainly performed by utilizing techniques such as blood drawing, amniocentesis or villus sampling, and the results obtained by amniocentesis and villus sampling are more accurate. However, amniocentesis and villus sampling are all invasive, which have a certain chance of causing miscarriage and even a risk to the mother's life.

### SUMMARY

The main purposes of the invention are to provide a cell sorting method and system which can sort out placental trophoblast cells from the cervical sample of a pregnant mammal for highly accurate prenatal fetal trisomy testing. In addition, the cervical sample is collected in a non-invasive way, avoiding risks such as miscarriage and harm to the pregnant mammal. The pregnant mammal is not restricted to human species; other mammals, such as pigs, cattle, horses, etc., are also suitable for the invention. The following illustrates examples of the number of days in pregnancy for several organisms: pigs: about 114 days; cattle: about 280 days; horses: about 335 to 342 days; dogs and cats: about 58 days to 70 days; rabbits: about 30 days to 33 days; white mice: about 21 days; hamsters: about 15 days to 18 days; guinea pigs: about 63 days to 68 days. The pregnancy of cattle is similar to that of humans, on which a sampling process can be performed during the 5^{th} week to the 20^{th} week of pregnancy, while a sampling process can be performed on pigs during about the 12^{th} day to the 54^{th} day of pregnancy, which are oriented by early sampling.

According to the above purposes, the invention provides a cell sorting method including: obtaining a cervical sample of a pregnant mammal, the cervical sample including placental trophoblast cells and cervical cells; removing mucus in the cervical sample; dispersing the placental trophoblast cells and the cervical cells; centrifuging the cervical sample to remove supernatant in the cervical sample; and using a dielectrophoretic chip to perform sorting on the cervical sample, so as to sort out the placental trophoblast cells from the cervical cells.

In accordance with one or more embodiments of the invention, the pregnant mammal is a pregnant woman.

In accordance with one or more embodiments of the invention, the cervical sample is collected at which a pregnancy of the pregnant mammal is 5^{th} week to 20^{th} week.

In accordance with one or more embodiments of the invention, using the dielectrophoretic chip to sort out the placental trophoblast cells and the cervical cells is performed in an environment of about 4° C.

In accordance with one or more embodiments of the invention, the cell sorting method further includes fixing the cervical sample by using a reservoir.

In accordance with one or more embodiments of the invention, the cell sorting method further includes: solving the cervical sample in a conductive solution after removing supernatant in the cervical sample, such that a cell density of the cervical sample achieves about 2×10⁵ cells/ml to 5×10⁵ cells/ml and that a conductivity of the cervical sample achieves less than 50 μS/cm.

In accordance with one or more embodiments of the invention, the conductive solution includes 0.25-0.5% bovine serum albumin (BSA).

In accordance with one or more embodiments of the invention, the conductive solution is a sucrose solution.

In accordance with one or more embodiments of the invention, the molar concentration of the sucrose solution is about 200 mM to 300 mM.

In accordance with one or more embodiments of the invention, the cell sorting method further includes: using a conductive solution with a conductivity of less than 10 μS/cm wash the cervical sample after removing the supernatant; and centrifuging the cervical sample again to further remove the supernatant.

According to the above purposes, the invention further provides a cell sorting system which includes a light-induced dielectrophoretic chip, a projection module and a power supply. The light-induced dielectrophoretic chip is used to generate an internal electric field to perform sorting on a cervical sample of a pregnant mammal, so as to sort out placental trophoblast cells and cervical cells in the cervical sample. The projection module is used to project patterned light towards the light-induced dielectrophoretic chip, such that the light-induced dielectrophoretic chip produces an light-induced effect to change the internal electric field, thereby sorting out the placental trophoblast cells and the cervical cells. The power supply is used to provide power to the light-induced dielectrophoretic chip with a frequency of about 20 KHz to 70 KHz, in order for the light-induced dielectrophoretic chip to generate the internal electric field. The power supply is provided with a peak voltage of about 10 V to 50 V for the cervical sample that has been fixed, and wherein the power supply is provided with a peak voltage of about 6 V to 15 V for the cervical sample that has not been fixed.

In accordance with one or more embodiments of the invention, the ratio of the resistance of a bright area of the light-induced dielectrophoretic chip to the resistance of a dark area of the light-induced dielectrophoretic chip is less than or equal to ⅕.

In accordance with one or more embodiments of the invention, the resistance of the bright area of the light-induced dielectrophoretic chip is less than or equal to 10Ω, and the resistance of the dark area of the light-induced dielectrophoretic chip is greater than or equal to 50Ω.

In accordance with one or more embodiments of the invention, the pregnant mammal is a pregnant woman, and the cervical sample the cervical sample is collected from a cervical portion of the pregnant woman at which a pregnancy of the pregnant mammal is 5^{th} week to 20^{th} week.

In accordance with one or more embodiments of the invention, the cell sorting system further includes a temperature controller that is used to control temperature of an environment where the light-induced dielectrophoretic chip is present at about 4° C. during the sorting of the cervical sample by the cell sorting system.

In accordance with one or more embodiments of the invention, the projection module includes a light emitting element and a light modulator. The light emitting element is used to generate light. The light modulator is used to convert the light into the patterned light.

In accordance with one or more embodiments of the invention, the light modulator is a digital micromirror device (DMD) or a liquid crystal on silicon (LCoS) device.

### BRIEF DESCRIPTION OF THE DRAWINGS

The foregoing aspects and many of the accompanying advantages of this invention will become more readily appreciated as the same becomes better understood by reference to the following detailed description, when taken in conjunction with the accompanying drawings.

FIG. 1 is a flowchart of a cell sorting method in accordance with some embodiments of the invention.

FIG. 2A and FIG. 2B are schematic diagrams of a cell sorting system in accordance with some embodiments of the invention.

FIG. 3A is a schematic diagram of a light-induced dielectrophoretic chip in accordance with some embodiments of the invention.

FIG. 3B and FIG. 3C illustrate planar views of the channel layer in FIG. 3A in accordance with various exemplarily example.

FIG. 4A and FIG. 4B respectively illustrate cross-sectional views respectively showing an electric field distribution in the light-induced dielectrophoretic chip 300 non-illuminated and illuminated by patterned light.

### DETAILED DESCRIPTION

The spirit of the disclosure is clearly described hereinafter accompanying with the drawings and detailed descriptions. After realizing preferred embodiments of the disclosure, any persons having ordinary skill in the art may make various modifications and changes according to the techniques taught in the disclosure without departing from the spirit and scope of the disclosure.

It will be understood that, although the terms “first” and “second” and may be used herein to describe various elements, components, regions, layers and/or sections, these elements, components, regions, layers and/or sections should not be limited by these terms. These terms are only used to distinguish one element, component, region, layer or section from another.

Terms used herein are only used to describe the specific embodiments, which are not used to limit the claims appended herewith. Unless limited otherwise, the term “a,” “an,” “one” or “the” of the single form may also represent the plural form.

Further, the spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

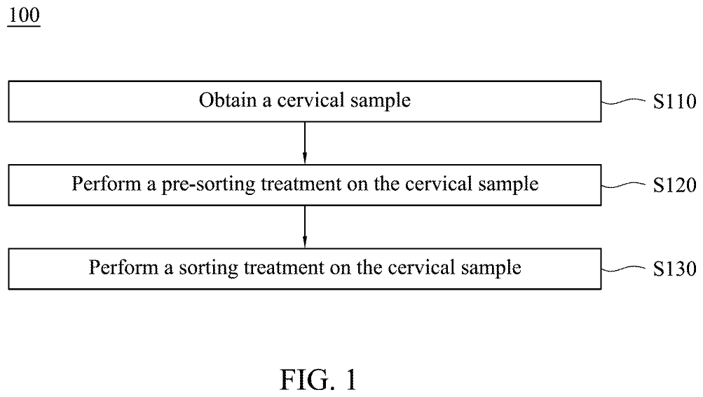

Referring to FIG. 1, FIG. 1 is a flowchart of a cell sorting method 100 in accordance with some embodiments of the invention. The cell sorting method 100 is used to detect the cervical sample collected from a pregnant mammal, so that professional medical personnel, such as doctors and medical laboratory scientist, can perform diagnosis or related analysis based on the test results.

It is noted that the cell sorting method 100 may be applied to a variety of mammalian species, which are not limited to human species. The cell sorting method 100 is also applicable for other mammals, such as pigs, cattle, horses, etc. The following illustrates examples of the number of days in pregnancy for several organisms: pigs: about 114 days; cattle: about 280 days; horses: about 335 days to 342 days; dogs and cats: about 58 days to 70 days; rabbits: about 30 days to 33 days; white mice: about 21 days; hamsters: about 15 days to 18 days; guinea pigs: about 63 days to 68 days. The pregnancy of cattle is similar to that of humans, on which a sampling process can be performed during the 5^{th} week to the 20^{th} week of pregnancy, while a sampling process can be performed on pigs during about the 12^{th} day to the 54^{th} day of pregnancy, which are oriented by early sampling. For convenience of description, the following embodiments are exemplified by pregnant women, but these embodiments are also applicable to the pregnant mammals other than humans.

First, Step S110 is performed to collect a cervical sample from a cervical portion of a pregnant woman. The collected cervical sample includes placental trophoblast cell and cervical cells. Tools for collecting the cervical sample may be non-invasive, such as cytobrush, ayre spatula, cervical brush, cytopick, but are not limited thereto. Furthermore, the sampling process may be performed on the pregnant woman during the 5^{th} week to the 20^{th} week of pregnancy to collect the cervical sample by gently inserting a non-invasive tool into the cervix in a clockwise or counterclockwise direction and then gently scraping the cells at the junction of squamous and columnar epidermal cells, in order for safely and smoothly collecting effective cervical cells. A cotton swab or another medical device may be used to remove excess mucus on the surface of the cervix before the cervical sample is collected for benefiting subsequent processes on the cervical sample.

After the cervical sample is collected, Step S120 is proceeded to perform a pre-sorting treatment on the cervical sample, which includes sample fixing, mucus removing and/or supernatant removing. The processes of Step S120 will be described below.

For the fixing treatment on the sample, a 10 ml reservoir with a mess fraction of methanol of about 30% to 60% may be used to fix the cervical sample at 4° C. for about 30 minutes to 60 minutes, such that cells of the cervical sample maintain intact without leaking their contents to maintain their conductivity and prolong their storage time. Another reservoir, such as ethanol, acetone, or the like, may also be used to fix the cervical sample.

The collected cervical sample usually includes mucus that easily adheres the placental trophoblast cells and cervical cells in the cervical sample into aggregations, which may affect the effect of cell sorting, and thus the mucus may be removed from the cervical sample first for subsequent procedures. For example, a mucolytic solution may be used to dissolve the mucus in the cervical sample, in order to avoid the cells the cervical sample being adhered into aggregations. The mucolytic solution may include acetylcysteine with a concentration of 20 mg/ml for the cervical sample to be placed at 37° C. for about 20 minutes to 45 minutes. However, the invention is not limited thereto; another suitable mucolytic solution, such as dithiothreitol, glacial acetic acid, or the like, may also be used to dissolve the mucus in the cervical sample, and the processing time and/or the contraction of the mucolytic solution may also be adjusted depending on the type of the mucolytic solution and/or the consistency of the mucus.

After the mucus dissolving treatment, a sieve with a pore diameter of about 70 μm to 100 μm may be used to further disperse the placental trophoblast cells and the cervical cells in the cervical sample, and cell counting is performed to determine if the number of collected cells is adequate.

Afterwards, the cervical sample is centrifuged to remove the supernatant. The rotational speed of the centrifuge used for centrifuging the cervical sample may be set to be 300 G to 400 G, and the duration of the centrifuging treatment is about 5 minutes to 10 minutes. After initially removing the supernatant, a further 10 ml conductive solution (of which the conductivity is less than 10 μS/cm) may be used to wash the cervical sample, and the centrifuging treatment is performed again to further remove the supernatant. After the centrifuging treatment, the cervical sample is redissloved in a conductive solution with 0.25-0.5% bovine serum albumin (BSA) and is preserved at 4° C. to avoid cell death, such that the cell density of the cervical sample achieves 2×10⁵ cells/ml to 5×10⁵ cells/ml and that the conductivity of the cervical sample achieves less than 50 μS/cm. The conductive solution may be a sucrose solution or another monosaccharide or disaccharide solution, of which the molar concentration is about 200 mM to 300 mM, and may also be used as a nutrient solution for the cells.

Step S120 may be completely or partially performed depending on different conditions. For example, if living cells are desired to be collected for subsequent cell sorting and analysis, the sample fixing treatment may not be performed to avoid loss of cell activity. If the amount of mucus in the cervical sample is little and the degree of cell adhesion into aggregations is slight, the mucolytic solution may not be used to solve the mucus in the cervical sample.

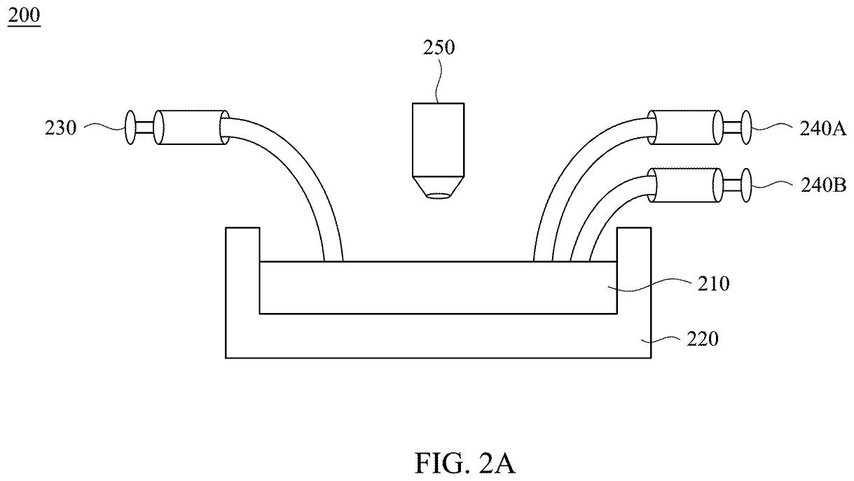

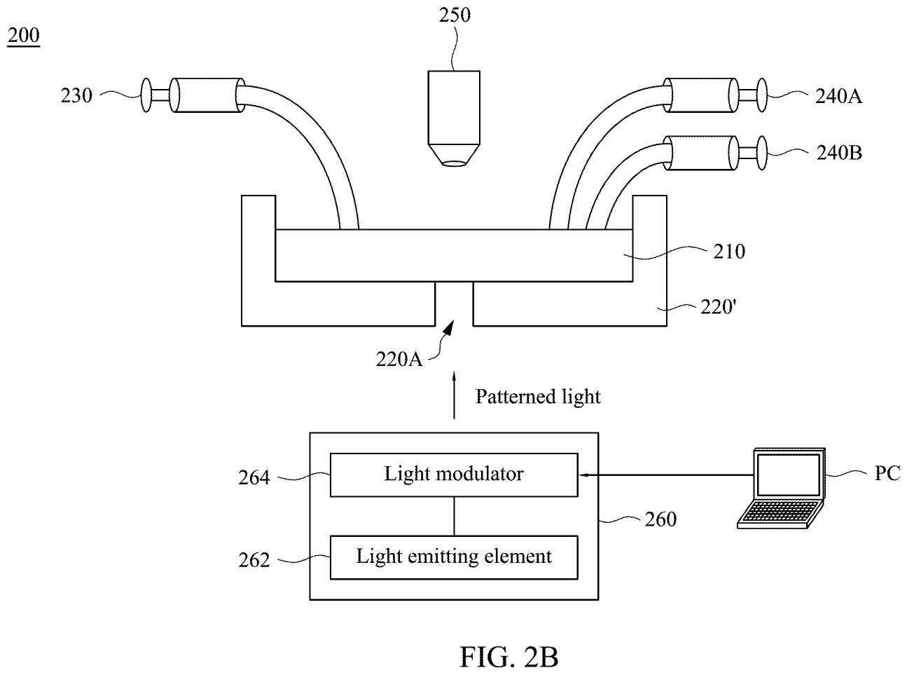

After the pre-sorting treatment step (i.e. Step S120) is completed, Step S130 is then performed, in which a dielectrophoretic chip is used to perform sorting on the cervical sample. FIG. 2A is a schematic diagram of a cell sorting system 200 in accordance with some embodiments of the invention. The cell sorting system 200 includes a dielectrophoretic chip 210, a supporting platform 220, an injector 230, accumulators 240A, 240B and an image detection module 250.

The dielectrophoretic chip 210 is used to sort the placental trophoblast cells and the cervical cells in the fluid. The dielectrophoretic chip 210 is used to generate an internal electric field, such that the placental trophoblast cells and the cervical cells move to different areas according to different dielectrophoresis (DEP) forces. As such, the placental trophoblast cells and the cervical cells can be sorted out by the dielectrophoretic chip 210. Step S130 may be performed in an environment of 4° C. That is, the dielectrophoretic chip 210 performs sorting at 4° C. to prolong the storage time of the cells.

The supporting platform 220 is used to support the dielectrophoretic chip 210. In some embodiments, the supporting platform 220 includes an accommodating structure for accommodating and fixing the dielectrophoretic chip 210. The accommodating structure may be a ring-shaped protruding structure, a rectangular-shaped recessing structure, a latch structure, or any other structure suitable for fixing the dielectrophoretic chip 210.

The injector 230 is connected to an inlet interface IN of the dielectrophoretic chip 210 for injecting a solution with placental trophoblast cells and cervical cells into the dielectrophoretic chip 210. The injector 230 may include a pump or another element that can control the speed of the fluid injected into the light-induced dielectrophoretic chip 210. In some embodiments, the injector 230 injects the solution into the dielectrophoretic chip 210 in a flow rate of 3 μl/min to 6 μl/min. The accumulators 240A, 240B are respectively connected to two outlet interfaces OUT1, OUT2 of the dielectrophoretic chip 210, for accumulating the placental trophoblast cells and the cervical cells sorted by the dielectrophoretic chip 210.

The image detection module 250 is disposed on the dielectrophoretic chip 210 for a user to measure the sorting status in the dielectrophoretic chip 210. In some embodiments, the image detection module 250 may include an image processing unit that performs image analyzing on captured images of sorting statuses to generate an analyzing result, and may real-time adjusting parameters of the cell sorting system 200 (such as the flow rate of the fluid injected by the injector 230 or another parameter that can be adjusted) depending on the analyzing result. In another embodiment, the image detection module 250 may be coupled to an entity with an image analysis function (e.g. a computer device PC shown in FIG. 2B), ant the abovementioned steps of image analysis may be performed in this entity.

In the invention, the dielectrophoretic chip 210 may be a light-induced dielectrophoretic chip or another chip in which DEP forces can be generated to sort out different cells. If the dielectrophoretic chip 210 is a light-induced dielectrophoretic chip, then as shown in FIG. 2B, the cell sorting system 200 may further include a projection module 260 disposed under the dielectrophoretic chip 210 for generating patterned light, and the supporting platform 220′ has an opening 220A for the patterned light to pass therethrough and project onto the dielectrophoretic chip 210. The luminous exitance and the wavelength of the patterned light generated by the projection module 260 may be between 9×10⁴ lux and 1.2×10⁵ lux and between 380 nm and 1400 nm, respectively. The projection module 260 includes a light emitting element 262 and a light modulator 264. The light emitting element 262 is used to generate a light, and may be, for example, a lamp, a light emitting diode (LED) or a laser, but is not limited thereto. For example, the light emitting element 262 may be a LED for emitting a light including components in a wavelength range visible light. The light modulator 264 converts the light generated by the light emitting element 262 into patterned light, and projects the patterned light onto the cell sorting region of the dielectrophoretic chip 210. In some embodiments, the light modulator 264 is a digital micromirror device (DMD) or a liquid crystal on silicon (LCoS) device used for receiving the light emitted by the light emitting element 262 and converting the light into patterned light according to the image data.

The projection module 260 may be communicatively connected to the computer device PC for receiving image data and determining the patterned light to be outputted accordingly. In detail, the projection module 260 may be communicatively connected to the computer device PC through wired communication technology (such as VGA, HDMI, eDP and USB) or wireless communication technology (such as WiFi and Bluetooth); the computer device PC transmits image data to the projection module 260, and then the light modulator 264 converts the light emitted by the light emitting element 262 into patterned light according to the image data. In addition, the image detection module 250 and the projection module 260 may be coupled to the same computer device PC. In some embodiments, the projection module 260 may further include a lens and/or a reflector for adjusting the focus and/or the planar range of the patterned light.

Further, in the example in which the dielectrophoretic chip 210 is a light-induced dielectrophoretic chip, a further lens (not shown) may be arranged between the dielectrophoretic chip 210 and the projection module 260 for adjusting the projection size on the dielectrophoretic chip 210 The magnification of the lens (not shown) may be determined depending on the architecture of the cell sorting system 200, e.g. the distance between the dielectrophoretic chip 210 and the projection module 260, the structure of the fluidic layer 340 in the dielectrophoretic chip 210 and/or the luminous exitance of the projection module 260. The lens (not shown) may be arranged in the opening 220A of the supporting platform 220, or between the dielectrophoretic chip 210 and the opening 220A, or between the opening 220A and the projection module 260.

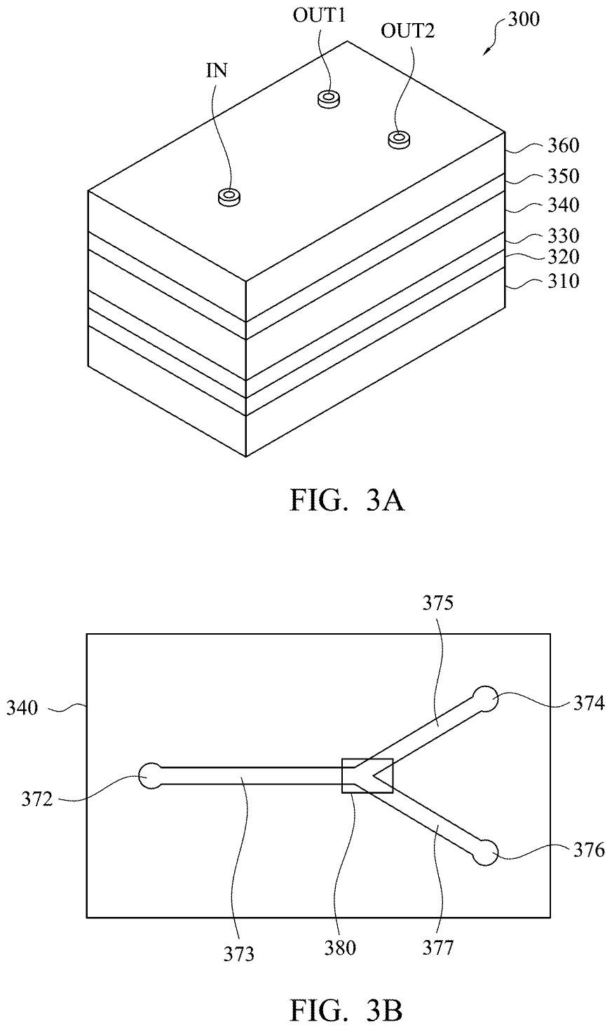

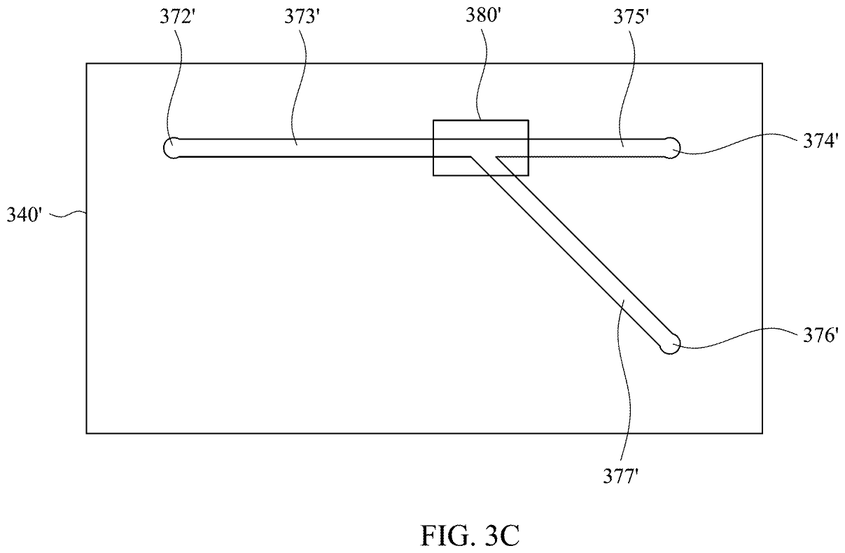

FIG. 3A is a schematic diagram of a light-induced dielectrophoretic chip 300 in accordance with some embodiments of the invention. The light-induced dielectrophoretic chip 300 may be an exemplarily example of the dielectrophoretic chip 210 in FIG. 2. The light-induced dielectrophoretic chip 300 includes a first substrate 310, a first electrode layer 320, a semiconductor layer 330, a fluidic layer 340, a second electrode layer 350 and a second substrate 360. The first substrate 310 is an optically transparent substrate, such as a glass substrate or a plastic substrate, but is not limited thereto.

The first electrode layer 320 is disposed on the first substrate 310, and includes transparent conductive material such as indium tin oxide (ITO), indium zinc oxide (IZO) or another similar conductive material.

The semiconductor layer 330 is disposed on the first electrode layer 320, and may indirect bandgap material such as silicon, germanium or another similar material. In addition, the semiconductor layer 330 may be formed of amorphous silicon, monocrystalline silicon, nanocrystalline silicon, polycrystalline silicon, or a combination thereof.

The fluidic layer 340 is disposed on the semiconductor layer 330 for guiding liquid with cells. FIG. 3B illustrates a planar view of the channel layer 340 in accordance with one exemplarily example. As shown in FIG. 3B, the fluidic layer 340 defines an inlet opening 372, an injecting region 373, a first outlet opening 374, a first accumulating region 375, a second outlet opening 376 and a second accumulating region 377. The injecting region 373, the first accumulating region 375 and the second accumulating region 377. The injecting region 373, the first accumulating region 375 and the second accumulating region 377 intersect in a projection area 380. The fluid is injected into the channel layer 340 through the inlet opening 372. The injecting region 373 is used to guide the fluid to the projection area 380. If the projection area 380 is under illumination by the light pattern, the internal electric field between the first electrode layer 320 and the second electrode layer 350 changes accordingly, such that the placental trophoblast cells and the cervical cells of the fluid move in different directions. Then, the first accumulating region 375 may guide the sorted placental trophoblast cells to flow to outside of the light-induced dielectrophoretic chip 300 through the first outlet opening 374, and the second accumulating region 377 may guide the sorted cervical cells to flow to outside of the light-induced dielectrophoretic chip 300 through the second outlet opening 376. Alternatively, depending on the pattern of the patterned light, the first accumulating region 375 may guide the sorted cervical cells to flow to outside of the light-induced dielectrophoretic chip 300 through the first outlet opening 374, and the second accumulating region 377 may guide the sorted placental trophoblast cells to flow to outside of the light-induced dielectrophoretic chip 300 through the second outlet opening 376.

The second electrode layer 350 is disposed on the fluidic layer 340. In some embodiments, the second electrode layer 350 includes transparent conductive material, such as indium tin oxide (ITO), indium zinc oxide (IZO) or another similar conductive material. In this embodiment, an external power source is coupled to the first electrode layer 320 and the second electrode layer 350 for providing a voltage difference between the first electrode layer 320 and the second electrode layer 350, so as to generate an internal electric field between the first electrode layer 320 and the second electrode layer 350.

The second substrate 360 is disposed on the second electrode layer 350. The second substrate 360 is an optically transparent substrate, such as a glass substrate or a plastic substrate, but is not limited thereto. In addition, an inlet interface IN and outlet interfaces OUT1 and OUT2 are disposed on the second substrate 360. The inlet interface IN is used to provide a path for the fluid to be injected into the inlet interface 372, and the outlet interface OUT1 and the outlet interface OUT2 are used to provide paths for different cell to flow to out of the light-induced dielectrophoretic chip 300 respectively from the first outlet opening 374 and the second outlet opening 376.

In some embodiments, the thickness of each of the first substrate 310 and the second substrate 360 is about 0.7 mm, the thickness of each of the first electrode layer 320 and the second electrode layer 350 is about 50 nm to 500 nm, the thickness of the semiconductor layer 330 is about 1 μm to 2 μm, and the thickness of the fluidic layer 340 is about 30 μm to 100 μm. In addition, in some embodiments, the angle between the injecting region 373 and the first accumulating region 375 is about 169 degrees, and the angle between the first accumulating region 375 and the second accumulating region 377 is about 22 degrees; the width of each of the injecting region 373, the first accumulating region 375 and the second accumulating region 377 is about 0.8 mm to 20 mm, and the caliber of each of the inlet opening 372, the first outlet opening 374 and the second outlet opening 376 is about 1.1 mm. In some embodiments, the size of the projection area 380 is approximately between 1×1 mm² and 10×10 mm². The values such as thickness, width and/or angle of each part in the light-induced dielectrophoretic chip 300 can be adjusted according to actual needs, and are not limited to the above values.

FIG. 3C is a planar view of a fluidic layer 340′ in accordance with another embodiment of the invention. The inlet opening 372′, the injecting region 373′, the first outlet opening 374′, the first accumulating region 375′, the second outlet opening 376′, the second accumulating region 377′ and the projection area 380′ of the fluidic layer 340′ respectively correspond to the inlet opening 372, the injecting region 373, the first outlet opening 374, the first accumulating region 375, the second outlet opening 376, the second accumulating region 377 and the projection area 380 shown in FIG. 3B. The difference between the fluidic layers 340, 340′ is that, the injecting region 373′ and the first accumulating region 375′ belong to the same direction, and the injecting region 373′ and the second accumulating region 377′ have a bending angle therebetween. The pattern of the patterned light can be designed according to the planar pattern of the fluidic layer 340′, such that the placental trophoblast cells and the cervical cells can be sorted out and flow into different accumulating regions.

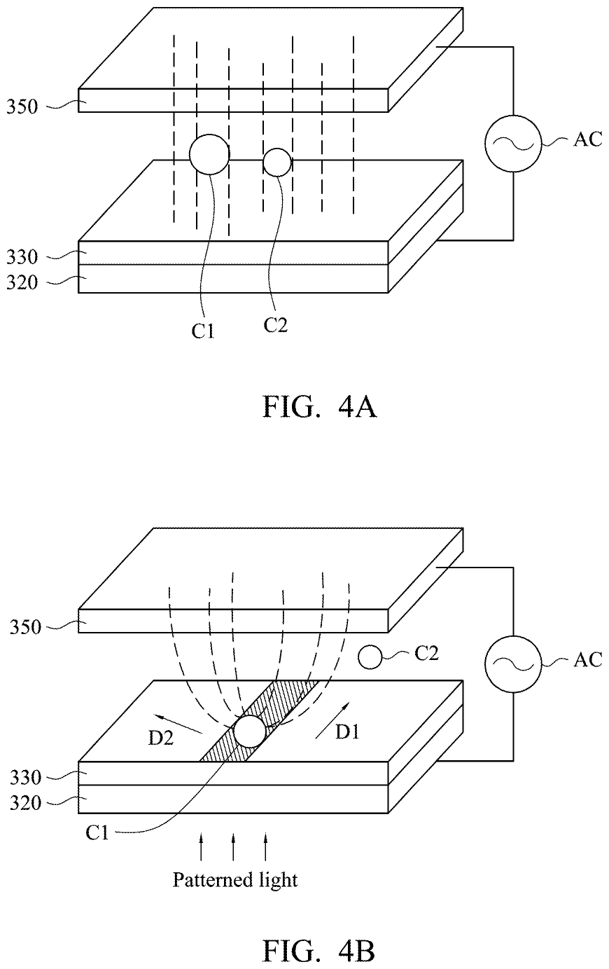

FIGS. 4A and 4B respectively illustrate cross-sectional views respectively showing an electric field distribution in the light-induced dielectrophoretic chip 300 non-illuminated and illuminated by patterned light. As shown in FIG. 4A, when the light-induced dielectrophoretic chip 300 is not illuminated by patterned light and the first electrode layer 320 and the second electrode layer 350 are electrically connected to the two terminals of a power supply AC, respectively, the electric field between the first electrode layer 320 and the second electrode layer 350 is uniform, and thus the cervical cells C1 and the placental trophoblast cells C2 are neither affected by the non-uniform electric field to move in particular directions. The frequency of the power provided by the power supply AC may be 20 KHz to 70 KHz. If the cervical sample has been fixed in Step S120, the peak voltage of the power provided by the power supply AC may be 10 V to 50 V; if the cervical sample has not been fixed in Step S120, the peak voltage of the power provided by the power supply AC may be decreased to be about 6 V to 15 V. In addition, the rate of the fluid with a cervical sample injected into the light-induced dielectrophoretic chip 300 may be 3 μl/min to 6 μl/min.

As shown in FIG. 4B, when illuminated by patterned light, the light-induced dielectrophoretic chip 300 produces an light-induced effect to change the electric field distribution between the first electrode layer 320 and the second electrode layer 350, such that the cervical cells C1 and the placental trophoblast cells C2 are separated by the DEP forces at the projection area 380. In particular, the cervical cells C1 move to the illuminated area of the pattern light by a positive DEP force D1, and the placental trophoblast cells C2 move to out of the illuminated area of the light pattern by a negative DEP force D2.

During the sorting of the cervical cells C1 and the placental trophoblast cells C2, a ratio of the resistance of a bright area of the light-induced dielectrophoretic chip 300 to the resistance of a dark area of the light-induced dielectrophoretic chip 300 is less than or equal to ⅕, in order to improve the sorting performance of the cervical cells C1 and the placental trophoblast cells C2. In some embodiments, the resistance of the bright area of the light-induced dielectrophoretic chip is less than or equal to 10Ω, and the resistance of the dark area of the light-induced dielectrophoretic chip is greater than or equal to 50Ω.

After sorting out placental trophoblast cells according to the above embodiments, because the rarity of placental trophoblast cells is limited, in order to obtain more information from a small amount of samples, in some embodiments, a whole genome amplification (WGA) treatment is performed the cells are subjected to before the genetic testing to increase the number of limited DNA samples, and then a genetic sequencing technique (e.g. by using a next-generation sequencer) is applied for the genetic testing to determine whether the fetus has chromosome aneuploidy, chromosome fragment deletion/repetition and/or single-gene diseases based on the test results, such as Down syndrome, Williams-Beuren syndrome, thalassemia, and/or the like. Alternatively, if the purity of the sorted placental trophoblast cells is high (e.g. greater than 70%), a gene chips may also be used to detect gene gain and loss in a whole genome for genetic testing, such as Prader-Willi syndrome, Cri du chat syndrome or other rare diseases caused by chromosome fragment deletion/duplication.

The purity of placental trophoblast cells can be verified prior to the WGA to the placental trophoblast cells. First, the DNA of the separated cells is extracted, and fluorescence labeled polymerase chain reaction (PCR) primers are used to amplify plural short tandom repeat (STR) polymorphic loci. Next, high resolution capillary electrophoresis and fluorescence detection technology are used to separate the amplified fragments and determine their lengths for achieving the classification of polymorphic loci, and the purity of placental trophoblast cells is obtained through calculation.

For the cervical sample collected from a pregnant woman during the 5^{th} week to the 10^{th} week of pregnancy, the purity of the placental trophoblast cells sorted according to the above embodiments is about 30% to 50%; for the cervical sample collected from a pregnant woman during the 10^{th} week to the 15^{th} week of pregnancy, the purity of the placental trophoblast cells sorted according to the above embodiments is about 40% to 60%; for the cervical sample collected from a pregnant woman during the 15^{th} week to the 20^{th} week of pregnancy, the purity of the placental trophoblast cells sorted according to the above embodiments is about 50% to 70%. In comparison with the purity of the placental trophoblast cells obtained by blood sampling, the purity of the placental trophoblast cells sorted according to the above embodiments can be greatly improved, thereby increasing the accuracy of fetal trisomy testing.

Through the cell sorting method and system according to the invention, the cervical sample of a mother can be collected in a non-invasive way to avoid invasive risks, and placental trophoblast cells can be sorted out from the cervical sample for prenatal fetal trisomy testing. Moreover, the cell sorting method and system according to the invention can sort out placental trophoblast cells for highly accurate fetal trisomy testing. Further, in comparison with conventional cell sorting methods for placental trophoblast cell extraction, the cell sorting method and system according to invention have the advantage of low hardware cost.

Although the invention is described above by means of the implementation manners, the above description is not intended to limit the invention. A person of ordinary skill in the art can make various variations and modifications without departing from the spirit and scope of the invention, and therefore, the protection scope of the invention is as defined in the appended claims.

What is claimed is:

1. A cell sorting method, comprising:
obtaining a cervical sample of a pregnant mammal, the cervical sample including placental trophoblast cells and cervical cells;
removing mucus in the cervical sample;
dispersing the placental trophoblast cells and the cervical cells;
centrifuging the cervical sample to remove supernatant in the cervical sample; and
using a dielectrophoretic chip to perform sorting on the cervical sample, so as to sort out the placental trophoblast cells from the cervical cells.

2. The cell sorting method of claim 1, wherein the pregnant mammal is a pregnant woman.

3. The cell sorting method of claim 1, wherein the cervical sample is collected at which a pregnancy of the pregnant mammal is 5^{th} week to 20^{th} week.

4. The cell sorting method of claim 1, wherein using the dielectrophoretic chip to sort out the placental trophoblast cells and the cervical cells is performed in an environment of about 4° C.

5. The cell sorting method of claim 1, further comprising:
fixing the cervical sample by using a reservoir.

6. The cell sorting method of claim 1, further comprising:
solving the cervical sample in a conductive solution after removing supernatant in the cervical sample, such that a cell density of the cervical sample achieves about 2×10⁵ cells/ml to 5×10⁵ cells/ml and that a conductivity of the cervical sample achieves less than 50 μS/cm.

7. The cell sorting method of claim 6, wherein the conductive solution includes 0.25-0.5% bovine serum albumin (BSA).

8. The cell sorting method of claim 7, wherein the conductive solution is a sucrose solution.

9. The cell sorting method of claim 8, wherein a molar concentration of the sucrose solution is about 200 mM to 300 mM.

10. The cell sorting method of claim 1, further comprising:
using a conductive solution with a conductivity of less than 10 μS/cm wash the cervical sample after removing the supernatant; and
centrifuging the cervical sample again to further remove the supernatant.

11. A cell sorting system, comprising:
a light-induced dielectrophoretic chip configured to generate an internal electric field to perform sorting on a cervical sample of a pregnant mammal, so as to sort out placental trophoblast cells and cervical cells in the cervical sample;
a projection module configured to project patterned light towards the light-induced dielectrophoretic chip, such that the light-induced dielectrophoretic chip produces an light-induced effect to change the internal electric field, thereby sorting out the placental trophoblast cells and the cervical cells; and
a power supply configured to provide power to the light-induced dielectrophoretic chip with a frequency of about 20 KHz to 70 KHz, in order for the light-induced dielectrophoretic chip to generate the internal electric field;
wherein the power supply is provided with a peak voltage of about 10 V to 50 V for the cervical sample that has been fixed, and wherein the power supply is provided with a peak voltage of about 6 V to 15 V for the cervical sample that has not been fixed.

12. The cell sorting system of claim 11, wherein a ratio of a resistance of a bright area of the light-induced dielectrophoretic chip to a resistance of a dark area of the light-induced dielectrophoretic chip is less than or equal to ⅕.

13. The cell sorting system of claim 12, wherein the resistance of the bright area of the light-induced dielectrophoretic chip is less than or equal to 10Ω, and wherein the resistance of the dark area of the light-induced dielectrophoretic chip is greater than or equal to 50Ω.

14. The cell sorting system of claim 11, wherein the pregnant mammal is a pregnant woman, and the cervical sample the cervical sample is collected from a cervical portion of the pregnant woman at which a pregnancy of the pregnant mammal is 5^{th} week to 20^{th} week.

15. The cell sorting system of claim 11, further comprising:
a temperature controller configured to control temperature of an environment where the light-induced dielectrophoretic chip is at about 4° C. during the sorting of the cervical sample by the cell sorting system.

16. The cell sorting system of claim 11, wherein the projection module comprises:
a light emitting element configured to generate light; and
a light modulator configured to convert the light into the patterned light.

17. The cell sorting system of claim 16, wherein the light modulator is a digital micromirror device (DMD) or a liquid crystal on silicon (LCoS) device.
